(19) United States
(12) Patent Application Publication
(10) Pub. No.: US 2024/0395366 A1
(43) Pub. Date: Nov. 28, 2024

(54) INFORMATION PROCESSING SYSTEM, INFORMATION PROCESSING DEVICE, AND INFORMATION PROCESSING METHOD

(71) Applicants: Preferred Networks, Inc., Tokyo-to (JP); ENEOS Corporation, Tokyo (JP)

(72) Inventor: So TAKAMOTO, Tokyo-to (JP)

(73) Assignees: Preferred Networks, Inc., Tokyo-to (JP); ENEOS Corporation, Tokyo (JP)

(21) Appl. No.: 18/669,787

(22) Filed: May 21, 2024

(30) Foreign Application Priority Data
May 26, 2023 (JP) 2023-087285

(51) Int. Cl. G16C 20/30; G16C 20/70

(52) U.S. Cl. CPC G16C 20/30; G16C 20/70

(57) ABSTRACT

An information processing system includes at least a first information processing device and a second information processing device. The second information processing device configured to transmit data relating to an atomic structure to the first information processing device. The first information processing device configured to receive the data relating to the atomic structure from the second information processing device, input the data relating to the atomic structure into a first model, acquire first information based on an output from an intermediate layer of the first model, and transmit the first information to the second information processing device. Additionally, the second information processing device configured to input the first information received from the first information processing device into a second model to acquire a predetermined value for the atomic structure.

### CROSS REFERENCE TO THE RELATED APPLICATIONS

This application is based upon and claims the benefit of priority from the Japanese Patent Application No. 2023-087285, filed on May 26, 2023, the entire contents of which are incorporated herein by reference.

### FIELD

This disclosure relates to an information processing system, an information processing device, and an information processing method.

### BACKGROUND

A Neural Network Potential (NNP) is used as means for acquiring a physical property value such as energy of an atom, a molecule, or the like using a neural network model. This NNP is composed of a neural network model having one or more layers. This neural network model is eventually constructed as a network for acquiring energy of each atom and therefore can acquire information for acquiring information about some property relating to an atom, a molecule, or the like, a physical property value, or information for acquiring the physical property value in its intermediate layer.

The NNP may be provided in a form of Software as a Service (Saas). In the case where the NNP is provided as Saas, the processing of the NNP is executed in a server, and an output in the intermediate layer of the NNP is not generally provided to a client, but can be considered to include various information about an atomic structure input as above.

### BRIEF DESCRIPTION OF THE DRAWINGS

FIG. 1 is a diagram schematically illustrating an information processing system according to an embodiment.

FIG. 2 is a chart schematically illustrating a flow of processing of an information processing device according to an embodiment.

FIGS. 3 to 5 are charts each schematically illustrating a flow of processing of an information processing device according to an embodiment.

FIG. 6 is a diagram schematically illustrating an implementation example of an information processing device according to an embodiment.

### DETAILED DESCRIPTION

According to one embodiment, an information processing system includes at least a first information processing device and a second information processing device. The second information processing device configured to transmit data relating to an atomic structure to the first information processing device. The first information processing device configured to receive the data relating to the atomic structure from the second information processing device, input the data relating to the atomic structure into a first model, acquire first information based on an output from an intermediate layer of the first model, and transmit the first information to the second information processing device. Additionally, the second information processing device configured to input the first information received from the first information processing device into a second model to acquire a predetermined value for the atomic structure.

The problem to be solved by the embodiments of this disclosure can be a problem corresponding to the effects mentioned in the embodiments as examples of non-limiting problems in addition to the above problem. In other words, the problem corresponding to at least arbitrary one of the effects described in the explanation of the embodiments of this disclosure can be the problem to be solved in this disclosure.

Hereinafter, embodiments of the present invention will be explained with reference to the drawings. The drawings and the explanation of the embodiments are indicated as examples and are not intended to limit the present invention.

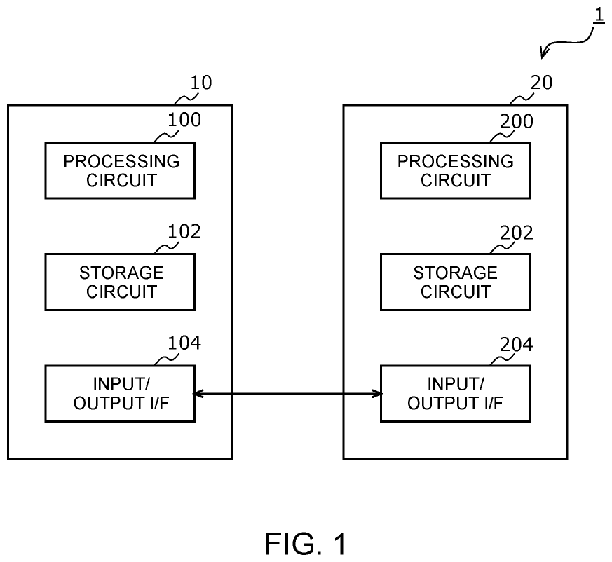

FIG. 1 is a diagram schematically illustrating an information processing system according to an embodiment. An information processing system 1 includes, for example, a first information processing device 10 and a second information processing device 20. Though one first information processing device 10 and one second information processing device 20 are illustrated in this drawing, the present invention is not limited to this.

The information processing system 1 may include, for example, one first information processing device 10 and a plurality of second information processing devices 20 connected to the first information processing device 10, or may include a plurality of first information processing devices 10 and a plurality of second information processing devices 20 each connected to any one first information processing device 10 of the plurality of first information processing devices 10. Besides, one second information processing device 20 may be provided for a plurality of first information processing devices 10.

In the case where the plurality of first information processing devices 10 are provided, the first information processing devices 10 may be connected with each other, and in the case where the plurality of second information processing devices 20 are provided, the second information processing devices 20 may be connected with each other. Further, the connection between arbitrary devices of the first information processing devices 10 and the second information processing devices 20 may be established, for example, by a line such as the Internet or may be directly established.

The first information processing device 10 includes a processing circuit 100, a storage circuit 102, and an input/output interface (hereinafter, described as an input/output I/F 104). The first information processing device 10 is a device which executes a predetermined arithmetic operation based on data received from the second information processing device 20 and transmits it to the second information processing device 20, and is, for example, a server. The first information processing device 10 can include control circuits which controls the respective configurations, power supplies which supply power to the respective configurations, and so on as appropriate.

The processing circuit 100 is a circuit which arithmetically processes data acquired by the first information processing device 10 via the input/output I/F 104. In the case where the information processing system 1 includes a plurality of first information processing devices 10, the processing circuits 100 of the plurality of first information processing devices 10 may perform an arithmetic operation in cooperation.

The storage circuit 102 is a circuit which stores information received via the input/output I/F 104. The processing circuit 100 refers to the data stored in the storage circuit 102 as needed. Further, the processing circuit 100 may store data in the middle of the arithmetic operation or data on an arithmetic operation result in the storage circuit 102 as needed.

The input/output I/F 104 is an interface which connects the inside and the outside of the first information processing device 10. The input/output I/F 104 may include an interface of an arbitrary standard.

The first information processing device 10 can provide SaaS, for example, to the second information processing device 20 which transmits data. The detailed arithmetic operation of the processing circuit 100 of the first information processing device 10 will be explained later.

The second information processing device 20 includes a processing circuit 200, a storage circuit 202, and an input/output I/F 204. The second information processing device 20 transmits data to be processed to the first information processing device 10, and receives a result of the arithmetic operation from the first information processing device 10, in order to use the service of the first information processing device 10. The second information processing device 20 may be, for example, a client corresponding to the first information processing device 10, and in this case, the information processing system 1 can operate as a server-client system. Further, the second information processing device 20 only needs to be a device different from the first information processing device 10, and may be, for example, a server.

The processing circuit 200 transmits data to be processed to the first information processing device 10 via the input/output I/F 204. Further, the processing circuit 200 can execute arbitrary processing on the arithmetic operation result received from the first information processing device 10 via the input/output I/F 204.

The storage circuit 202 can store the arithmetic operation result by the processing circuit 200 of the second information processing device 20, data to be transmitted to the first information processing device 10, or the data received from the first information processing device 10. The processing circuit 200 can refer to the data stored in the storage circuit 202 as needed.

The input/output I/F 204 is an interface which connects the inside and the outside of the second information processing device 20. The input/output I/F 204 may include an interface of an arbitrary standard.

Next, the operation of the information processing system 1 will be explained. The information processing system 1 executes the arithmetic processing in the processing circuit 100 of the first information processing device 10 based on the data to be processed transmitted from the second information processing device 20, and transmits a result of the execution of the arithmetic processing to the second information processing device 20.

The first information processing device 10 is a device which executes, for example, an arithmetic operation about the neural network for the received data and transmits its result. More specifically, the first information processing device 10 can receive data relating to an atomic structure being an input (for example, a scalar, a vector, a matrix of arbitrary dimensions, or a tensor or arbitrary dimensions, that includes information regarding to the atomic structure) of a model (for example, a model used for the NNP), and transmit a physical property value regarding the received atomic structure to the second information processing device 20. Further, the first information processing device 10 can also forward propagate the data relating to the received atomic structure up to an intermediate layer of a trained model of the model (for example, the model used for the NNP), and transmit an output from the intermediate layer to the second information processing device 20.

The data relating to the atomic structure may include at least information about the type of one or more atoms and the position of the atom (may be coordinates) as one example. For example, based on the data on the atomic structure, each of the information processing devices can acquire information on a chemical compound, an aggregate of molecules, or the like which is described by the atomic structure. Besides, as another example, the data relating to the atomic structure may include information on boundary conditions. Further, as another example, the data relating to the atomic structure may include at least information on an atomic nucleus (proton, neutron) and electron of one or more atoms.

The NNP includes a trained model which outputs a physical property value, for example, energy when the atomic structure is input thereinto. The trained model is formed including a Graph Neural Network (GNN) as a non-limiting example. Therefore, the output from the intermediate layer of the NNP is estimated to include some arithmetic operation result from the atomic structure to the physical property value such as energy.

Therefore, in this disclosure, the first information processing device 10 arithmetically operates at least a forward propagation result up to the intermediate layer before acquisition of the physical property value such as energy about the atomic structure received from the second information processing device 20, and transmits it to the second information processing device 20. The second information processing device 20 can continuously execute the processing related to the atomic structure based on the acquired output of the intermediate layer.

Here, the first information processing device 10 may have a processing circuit 100 sufficiently higher in arithmetic operation performance than the processing circuit 200 of the second information processing device 20. The processing circuit 100 is a circuit which is higher in parallel arithmetic operation performance than the processing circuit 200 and can execute high-speed arithmetic processing. Further, the processing circuit 100 is a circuit which can execute a predetermined arithmetic operation, for example, a graph arithmetic operation at a higher speed than the processing circuit 200. The processing circuit 100 of the first information processing device 10 is formed to be able to refer to the model about one or more NNPs, and arithmetically operates up to the output from the intermediate layer in response to the request from the second information processing device 20, and outputs it.

### First Embodiment

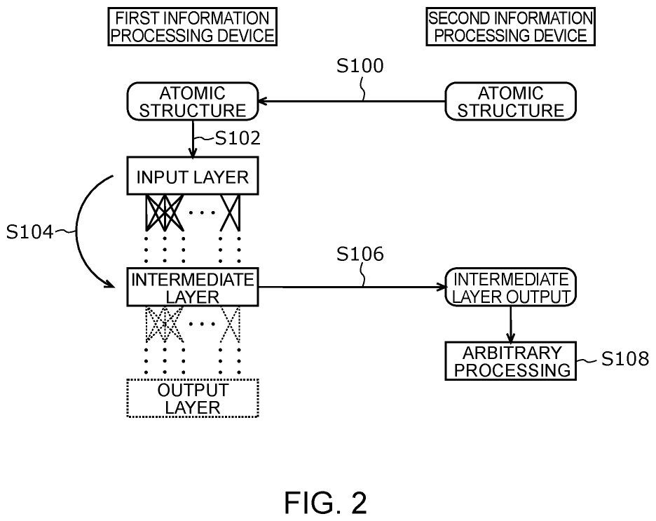

FIG. 2 is a chart schematically illustrating a flow of processing between the information processing devices of the information processing system 1 according to an embodiment.

Note that the trained model in the chart is illustrated as a model (NNP) connected from an input layer to an output layer via a plurality of intermediate layers but, not limited to this, may have a form including the input layer, one intermediate layer, and the output layer. Besides, the intermediate layer which outputs the information to be transmitted to the second information processing device 20 may be an intermediate layer next to the input layer or may be an intermediate layer just before the output layer.

As explained above, this model may be the GNN as an example, or may be another neural network model not limited to this. In this case, this model is the one trained by an appropriate method based on the type of the neural network model.

Further, this model is not limited to the neural network model but may be another model which performs an appropriate output with respect to an input. In this case, this model may be a model capable of outputting an intermediate value with respect to a final output of the model, or may be one or more models which belong to a model group for obtaining the final output by the plurality of models and for outputting an intermediate output. This also applies to the following embodiments.

The second information processing device 20 transmits an atomic structure data to be processed to the first information processing device 10 via the input/output I/F 204 (S100). The atomic structure data may be described, for example, in a graph format or may be described in a format other than this. Hereinafter, the atomic structure data may be the data relating to the atomic structure, or data which may be data extracted or converted from the data relating to the atomic structure into data suitable for input (for example, the graph format data or in the format other than this).

The first information processing device 10 acquires the atomic structure data from the second information processing device 20 and inputs it into the input layer of the trained model deployed in the first information processing device 10 (S102).

The first information processing device 10 forward propagates the atomic structure data from the input layer up to the intermediate layer to acquire an output (first information) from the intermediate layer (S104).

The first information processing device 10 transmits the output (first information) from the intermediate layer to the second information processing device 20 via the input/output I/F 104 (S106).

The second information processing device 20 can execute arbitrary processing based on the data acquired from the first information processing device 10 (S108).

As explained above, the information processing system 1 according to this embodiment can form a client-server system capable of acquiring the output from the intermediate layer of the NNP at high speed. The second information processing device 20 uses Saas provided by the first information processing device 10 and thereby can execute an arithmetic operation with high arithmetic operation cost in the processing of the NNP in the first information processing device 10 and acquire the output from the intermediate layer at high speed.

The first information processing device 10 may output the data up to a predetermined intermediate layer of a predetermined trained model to the second information processing device 20.

The first information processing device 10 can selectively use a plurality of trained models, in which case a trained model to be used is selected based on the data received from the second information processing device 20 and the result forward propagated up to the predetermined intermediate layer can be transmitted to the second information processing device 20.

In any of the above cases, the second information processing device 20 does not need to have the information about the trained model. In other words, the second information processing device 20 does not need to have the information for forming the same model as that of the first information processing device 10. As a matter of course, the second information processing device 20 and the first information processing device 10 share parameters and the like of the model to be used therein so that the trained model can be constructed also in the second information processing device 20.

In the case where the second information processing device 20 has at least part of the data relating to the trained model, the first information processing device 10 can also select the trained model to be used in response to the request from the second information processing device 20.

The first information processing device 10 can transmit a result of forward propagation of input data up to a requested intermediate layer to the second information processing device 20 based on a request, regarding from which intermediate layer of the trained model data is to be acquired, from the second information processing device 20. In this case, the user can designate up to which intermediate layer data is to be acquired, via the second information processing device 20. Further, the user can also designate which model is to be used from among the plurality of trained models.

The first information processing device 10 may transmit the information on the used trained model together with the output from the intermediate layer to the second information processing device 20. With this information on the model, the second information processing device 20 or the user can acquire the information on which trained model was used.

The first information processing device 10 may include, for example, identification information on the trained model (may include version information, revision information, and so on), information about the accuracy of numerical values, and information about a device to be operated. Further, the user may designate these pieces of information in the first information processing device 10 at timing when requesting the data.

### Second Embodiment

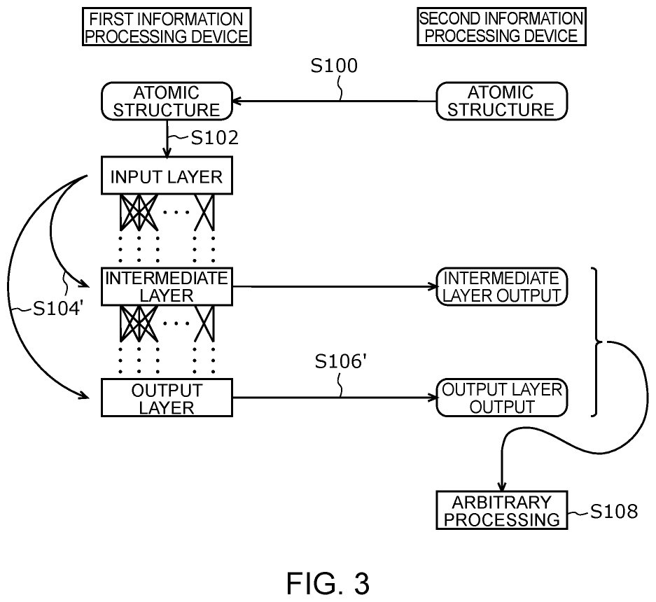

FIG. 3 is a chart schematically illustrating a flow of processing between the information processing devices of the information processing system 1 according to an embodiment.

In this embodiment, the first information processing device 10 forward propagates the received atomic structure to the trained model to acquire the output from the intermediate layer and an output (second information) from the output layer (S104′).

The first information processing device 10 outputs output data (second information) from the output layer, for example, typically an energy value for the atomic structure together with the output data from the intermediate layer acquired at S104′ (S106′).

The second information processing device 20 can acquire the output data from each of the intermediate layer and the output layer of the trained model in the NNP, and execute arbitrary subsequent processing (S108).

The second information processing device 20 can execute, for example, later-explained similarity calculation and classification of the atomic structure using information from both of the intermediate layer and the output layer as the arbitrary subsequent processing.

With this processing, the second information processing device 20 can execute processing further referring to the data from the output layer in addition to the above embodiment.

### Third Embodiment

The second information processing device 20 calculates not the atom coordinates themselves included in the atomic structure but the similarity in an intermediate layer space forming the NNP, based on the received output from the intermediate layer, and thereby can acquire the data on what type of atomic structure the atomic structure has a structure and property close thereto. Further, the second information processing device 20 can also use the similarity in the calculated intermediate layer space instead of or in addition to the atom coordinates included in the atomic structure.

The atomic structure transmitted by the second information processing device 20 and processed by the first information processing device 10 includes data representing the structure of an atomic aggregate (molecular aggregate). Therefore, the second information processing device 20 can acquire the similarity between the atomic structures in the intermediate layer by using the output from the intermediate layer of the trained model forming the NNP.

The second information processing device 20 can acquire the similarity as a whole molecule, for example, by executing graph matching for the set of the outputs from the intermediate layers. The second information processing device 20 can acquire the output value from the intermediate layer via the first information processing device 10, for example, about various molecular structures. The second information processing device 20 can acquire what atomic structures have close structures and properties by solving a combinational optimization problem of the thus-acquired various molecular structures.

Further, the second information processing device 20 can also infer the similarity between not the molecules or chemical compounds but the atoms using the output from the intermediate layer. Similarly to the above, the second information processing device 20 can infer the similarity between atoms by acquiring the output from the intermediate layer about the data on the atomic structures representing the atoms via the first information processing device 10.

For example, the second information processing device 20 can transmit the atomic structures representing various types of atoms to the first information processing device 10 to acquire the output from the intermediate layer. In this information processing system 1, the second information processing device 20 can present the user with the data having a structure and property close to a certain atom included in the atomic aggregate.

The output from the intermediate layer is information about the type of the atom and information about the bonding between the atoms, for example, data representing a type of each atom and the bonding between the atoms, as with the atomic structure to be input. Therefore, the second information processing device 20 can acquire the information about the types of the atoms and the structure and property based on the bonding state between the atoms by using the output from the intermediate layer.

The second information processing device 20 can also build a database based on the above or register the data in an existing database.

The second information processing device 20 can also present the user with a result of a simulation relating to an object represented by the input atomic structure based on the output from the intermediate layer.

Besides, the second information processing device 20 can also implement clustering in the intermediate layer space.

Note that the second information processing device 20 which has acquired the information from the first information processing device 10 calculates the similarity in the above, but the present invention is not limited to this. For example, the first information processing device 10 may calculate the similarity based on the output from the intermediate layer and the second information processing device 20 may receive the similarity. Besides, the first information processing device 10 can also execute processing related to the database, the processing related to the simulation, or processing related to the clustering.

### Fourth Embodiment

According to the information processing system 1 in this disclosure, it is also possible to realize potential fitting. The second information processing device 20 can use, for example, an output from an intermediate layer of a trained model (first model) deployed in the first information processing device 10 as an input of another trained model (second model).

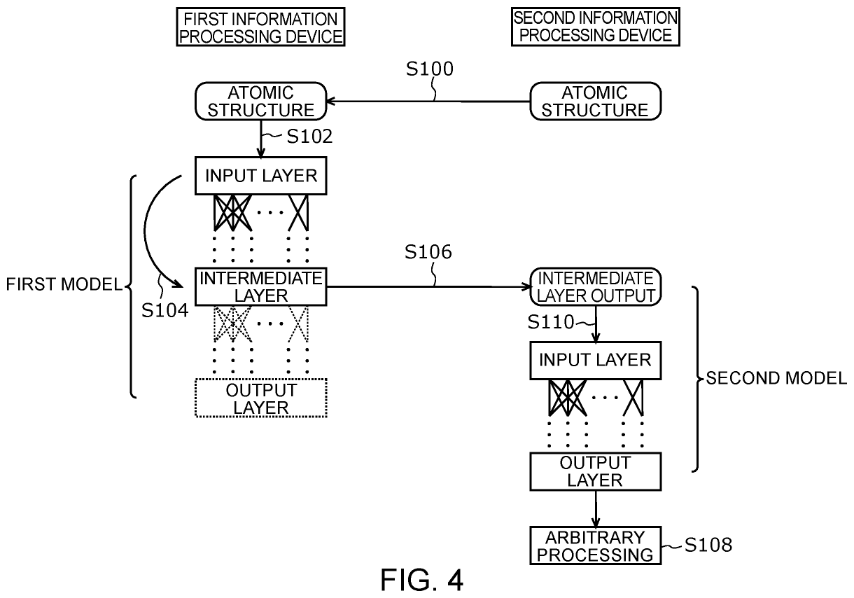

FIG. 4 is a chart schematically illustrating a flow of processing between the information processing devices of the information processing system 1 according to an embodiment.

The second information processing device 20 can acquire the output from the intermediate layer of the first model deployed in the first information processing device 10 (S106), and can use this output result as the input into the input layer of the second model deployed in the second information processing device 20 (S110).

The second information processing device 20 can input the output data into the input layer as illustrated in FIG. 4 by acquiring the output data from the intermediate layer from the first information processing device 10. The second information processing device 20 can further use the output data from the intermediate layer acquired in the first information processing device 10, for example, as learning data for a new model to be trained.

As a matter of course, in the case where the performance of the processing circuit 200 is not sufficient in the second information processing device 20, the intermediate layer output of the first model arithmetically operated by the first information processing device 10 is stored in the storage circuit 202 of the second information processing device 20 or an external storage circuit and the second information processing device 20 can transmit a request for training of the second model using another server. In this case, the other server and the first information processing device 10 are not limited to be separate servers, but the same information processing device may be used.

For example, the second information processing device 20 can acquire a characteristic value desired by the user by using the output from the intermediate layer of the first model in a versatile NNP appropriately trained with rich learning data which acquires energy from the atomic structure and using the second model which outputs a physical property value desired by the user. This physical property value to be acquired may be a characteristic value such as Highest Occupied Molecular Orbital (HOMO), Lowest Unoccupied Molecular Orbital (LUMO), or a polarizability as a non-limiting example.

The use of the information processing system 1 makes it possible to realize appropriate inferring processing or the like by using the first model in the first information processing device 10 which is versatile and can arithmetically operate with high accuracy and at high speed and using the second model which is desired to be secret from a third party.

The first information processing device 10 can also execute the training of the second model for the user and inform the user of parameters regarding the second model. In this manner, it is also possible to customize the trained model using the output from the intermediate layer of the first model and provide it as SaaS.

According to this embodiment, the information processing system 1 can provide the NNP specialized for a customer. The information processing system 1 can further realize fitting to data possessed by the customer. The parameters of the second model to be used for the fitting can be held in the second information processing device 20 being a terminal on the user side or held in the first information processing device 10.

Further, the second model may be a model which outputs energy as another example.

### Fifth Embodiment

The first information processing device 10 transmits the result of forward propagation of the trained model to the second information processing device 20 in each of the above embodiments, but the present invention is not limited to this. The first information processing device 10 may transmit a result of backward propagation through at least some layers of the trained model to the second information processing device 20.

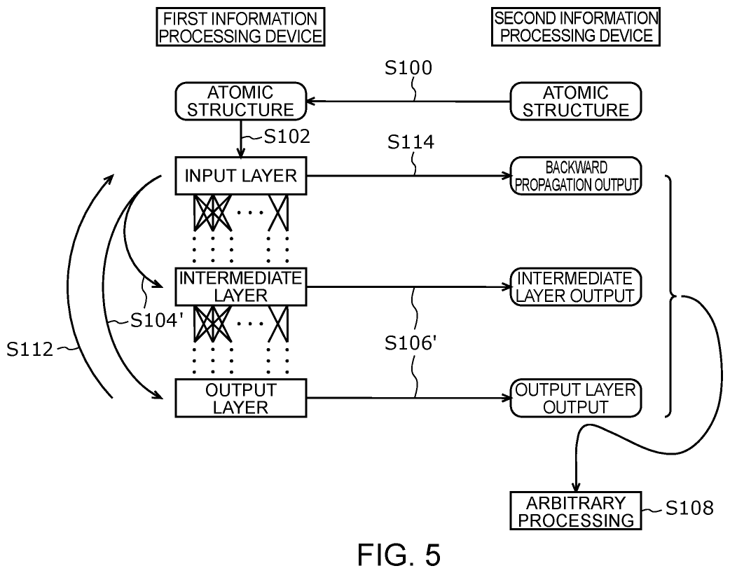

FIG. 5 is a chart schematically illustrating a flow of processing between the information processing devices of the information processing system 1 according to an embodiment.

The first information processing device 10 may perform backward propagation from the output layer or an arbitrary intermediate layer to an intermediate layer before it or the input layer after the forward propagation (S112), and transmit a backward propagation result to the second information processing device 20 (S114). As explained above, the first information processing device 10 may calculate a backward propagation output (third information) in an arbitrary layer or between arbitrary layers and transmit it together with the output from the intermediate layer to the second information processing device 20.

FIG. 5 illustrates a form in which the first information processing device 10 executes backward propagation processing, but the present invention is not limited to this. In the case where the second information processing device 20 is desired to execute backward propagation of the trained model, for example, the first information processing device 10 may transmit information which can be backward propagated of the intermediate layer output (or may include an output layer output) together with the intermediate layer output to the second information processing device 20.

As a simple example, the first information processing device 10 may transmit parameters of the trained model to the second information processing device 20. In this case, the second information processing device 20 can perform backward propagation processing by forming the trained model from the received parameters. The second information processing device 20 becomes able to perform the backward propagation processing, and thereby can also realize relearning of the trained model in the NNP and acquire a value of force with respect to an energy value (a value obtained by position differentiation of energy) as an example.

Further, as another example, an arbitrary neural network model (second model) formed on the second information processing device 20 side which uses the output of the intermediate layer of the trained model (first model) in the NNP as an input can also be trained on the first information processing device 10 side. In this case, it is possible to perform learning of the second model on the second information processing device 20 side based on the output from the intermediate layer of the first model and transmit learned parameters to the first information processing device 10 to thereby execute potential fitting.

Not limited to the above, during or at the end of training of the second model formed on the second information processing device 20, the first model can also be fitted (customized) using its result.

The second information processing device 20 can execute backward propagation processing, for example, from the output layer of the second model, and transmit a backward propagation value (fourth information) up to the input layer of the second model to the first information processing device 10 as a backward propagation value of the intermediate layer of the first model together with the information about the atomic structure. The first information processing device 10 can backward propagate the first model based on the information about the atomic structure, the backward propagation value received from the second information processing device 20, and the information on the first model, and execute arbitrary processing, for example, potential fitting of the first model based on the result of the second model.

Further, as another non-limiting example, the first information processing device 10 can perform backward propagation up to the input layer of the first model using the backward propagation value of the second model in response to a request from the second information processing device 20 and calculate the force acting on atoms.

The use of the output in this embodiment also enables the second information processing device 20 to acquire the information on the position differentiation or the like for each atom. For example, by using this result, the second information processing device 20 can optimize, for example, reaction path estimation or the like as a minimization problem of cost using the difference between intermediate layer outputs in certain two structures as the cost. The use of the information on position differentiation enables transformation of the differentiation of the value of the intermediate layer to information on a position (differentiation of a position), and output of a natural pathway interpolating between the two structures.

### Sixth Embodiment

The second information processing device 20 can infer various physical properties relating to the transmitted atomic structure by combining the output of the intermediate layer of the NNP and other data based on the result acquired from the first information processing device 10.

For example, the second information processing device 20 can infer various physical properties based on the values registered in the existing database or experimental values or the like. For example, the second information processing device 20 can infer various physical properties using a calculated value, a calculation result of other software, and a fingerprint. Further, for example, the second information processing device 20 can infer various physical properties based on the weight of each atom, atomic number, and the like.

In the case of capable of using a plurality of NNPs, the second information processing device 20 can also execute arbitrary processing using the output values from the intermediate layers in different NNPs. The different NNPs may be formed in different first information processing devices 10 or in the same first information processing device 10.

As another non-limiting example of this embodiment, the second information processing device 20 (or may be another second information processing device) can train the second model using the output from the intermediate layer of one or a plurality of NNPs and the above other data. The information processing device can train the second model by an arbitrary machine learning method, for example, using the output from the intermediate layer of the NNP and the other data as the input data and using known physical property values as teacher data.

### Seventh Embodiment

The second information processing device 20 can also generate visualized graphics based on the output from the intermediate layer in the NNP. The second information processing device 20 can visualize the output from the intermediate layer itself or a result obtained by processing the output (including the output input into the other trained model) by transforming it by two-dimensionally or three-dimensionally appropriate processing centered on some index.

The second information processing device 20 transforms a scalar value of an n-dimensional value×atomicity, for example, for each atom acquired as the intermediate layer output and thereby can plot it on a two-dimensional space or a three-dimensional space.

Further, the second information processing device 20 can realize processing such as comparison between atoms, comparison for each group of a predetermined number of atoms, comparison for each molecule, or comparison between a plurality of molecular structures.

Further, the second information processing device 20 can visualize the distribution of the output from the intermediate layer as a histogram. The visualization makes it possible to output display or the like in a space which the user can understand.

Specifically, the second information processing device 20 can calculate the distance between a plurality of molecular structures (atomic aggregates) from the intermediate layer output and plot this distance on the two-dimensional space. The second information processing device 20 can plot the distance on the two-dimensional space using the t-SNE (t-Distributed Stochastic Neighbor Embedding) as a non-limiting example. In addition, the second information processing device 20 can visualize the output result from the intermediate layer using an arbitrary method regarding display of a graph.

The visualization as above enables the user to visually confirm the result or the like of clustering an arbitrary physical property, for example, by the groups or atomic structures close in calculated similarity.

The second information processing device 20 can visualize information such as clustering of an oxide and a metal, for example, as a result of Bader charge analysis.

### Eight Embodiment

The second information processing device 20 can infer a potential parameter in the processing at S108 in FIG. 2 to FIG. 5. For example, as an example of FIG. 4, the second information processing device 20 can train the second model which acquires the potential parameter.

In this case, the second model is trained as a model which acquires the potential parameter from the output of the intermediate layer of the first model. The potential parameter is a simpler model than the NNP and is a parameter based on a so-called classical potential. More specifically, the potential parameter is a parameter including information on the length and strength of a spring when the bonding between atoms is expressed by the spring.

The second information processing device 20 trains the second model so that the output of the intermediate layer of the first model is input into the input layer of the second model and the potential parameter is output from the output layer. The value of the intermediate layer of the first model has various information about each atom in the atomic structure. Therefore, the formation of a model using the information on each atom as an input enables the second information processing device 20 to train the second model which acquires the potential parameter.

The second information processing device 20 repeats the input of the intermediate layer output of the first model into the input layer of the second model and the forward propagation and the backward propagation processing from the output layer of the second model so that the output becomes the potential parameter. As a result of this, the second information processing device 20 can train the second model which outputs the potential parameter when the intermediate layer output of the first model is input thereinto.

The second information processing device 20 performs training, for example, so that the inferred potential parameter matches a correct potential parameter. The correct potential parameter may be fitted using the one calculated using the NNP or using an already-known value.

As a concrete example, the potential parameter about a covalent crystal of carbon is largely different between diamond and graphite depending on the way of covalent bonding. In the case where information on carbon is input as the atomic structure, the intermediate layer of the NNP outputs data having characteristics of diamond when the atomic structure of diamond is input, and outputs data having characteristics of graphite when the atomic structure of graphite is input.

By using the output from the intermediate layer of the NNP, the above potential parameter can be acquired. The second information processing device 20 can train the second model using the potential parameter.

Note that it is also possible to connect this result to the potential fitting in the above embodiments.

According to each of the above embodiments, as the concrete example of applying the above disclosure, the information processing system 1 can realize the processing such as the similarity calculation of the atomic structure based on the value output from the intermediate layer of the NNP, the potential fitting, the prediction of the physical property value, and the visualization of the atomic structure and an amount about physical property value.

Though the value of the intermediate layer in the NNP is used in each of the explained embodiments, for example, the value of any one of the value output for each atom and the value output in a combination of atoms, or the values of both of them can be used as the output from the intermediate layer in the NNP. The selection of the value can be appropriately made depending on the amount desired to acquire using the output result of the intermediate layer.

The value output in the combination of atoms is, for example, a value corresponding to the whole atomic structure. The combination of atoms is typically, but not limited to, a combination of two types of atoms, and may be a combination of three or more types of atoms.

Further, the first information processing device 10 does not directly output the value of the intermediate layer of the NNP, but may output it to the second information processing device 20 via some transformation to the value of the intermediate layer.

The first information processing device 10 can output, for example, the value of the intermediate layer of the NNP via a function of linearly or nonlinearly transform it. The first information processing device 10 may multiply the value of the intermediate layer of the NNP by a constant and output the resulting value as a simple example.

Further, the first information processing device 10 can execute, for example, statistic processing on the value of the intermediate layer of the NNP and output the resulting value. The first information processing device 10 may output, for example, a result obtained by performing a principal component analysis (PCA).

The processing of linear or nonlinear function or the statistic processing may be realized by some model, for example, a trained neural network model. More specifically, the first information processing device 10 may input the output from the intermediate layer of the NNP into the trained model, and transmit the output of this trained model to the second information processing device 20.

In other words, the processing to be executed by the second information processing device 20 in each of the above embodiments may be executed by the first information processing device 10 and its result may be transmitted to the second information processing device 20.

As above, the first information processing device 10 can transmit not only raw data inferred by the NNP but also data obtained by performing some processing on the raw data, to the second information processing device 20.

Note that the second information processing device 20 is a device different from the first information processing device 10 in the above embodiments, but the present invention is not limited to this.

For example, the processing to be executed by the first information processing device 10 in the above embodiments may be provided as an application (software) executable in the second information processing device 20, and this application is executed in the second information processing device 20, whereby the processing executed by the first information processing device 10 in the above embodiments may be executable in the second information processing device 20.

In this case, the application may be an application which executes processing in a black box whose processing details are difficult to trace from the second information processing device 20. Besides, Application Programming Interface (API) may be provided on the application side, so that the user can use as appropriate the processing executed by the first information processing device 10 through the API in the second information processing device 20.

Besides, as another form, the first information processing device 10 executes the processing executed by the second information processing device 20 in the above embodiments, and the second information processing device 20 acquires its result. In this case, the first information processing device 10 can be used as the server of SaaS as in the above embodiments.

The first information processing device 10 may provide a user interface, and the user may request, from the second information processing device 20, the first information processing device 10 to execute the above processing executed in the second information processing device 20 using the user interface provided by the first information processing device 10. In addition, the user can describe a request in the first information processing device 10 in an arbitrary format, in which case the second information processing device 20 transmits the file in which the request of the user is described to the first information processing device 10 and thereby can make the first information processing device 10 execute desired processing.

The trained models of above embodiments may be, for example, a concept that includes a model that has been trained as described and then distilled by a general method.

Some or all of each device (at least one of the first information processing device 10 or the second information processing device 20) in the above embodiment may be configured in hardware, or information processing of software (program) executed by, for example, a CPU (Central Processing Unit), GPU (Graphics Processing Unit). In the case of the information processing of software, software that enables at least some of the functions of each device in the above embodiments may be stored in a non-volatile storage medium (non-volatile computer readable medium) such as CD-ROM (Compact Disc Read Only Memory) or USB (Universal Serial Bus) memory, and the information processing of software may be executed by loading the software into a computer. In addition, the software may also be downloaded through a communication network. Further, entire or a part of the software may be implemented in a circuit such as an ASIC (Application Specific Integrated Circuit) or FPGA (Field Programmable Gate Array), wherein the information processing of the software may be executed by hardware.

A storage medium to store the software may be a removable storage media such as an optical disk, or a fixed type storage medium such as a hard disk, or a memory. The storage medium may be provided inside the computer (a main storage device or an auxiliary storage device) or outside the computer.

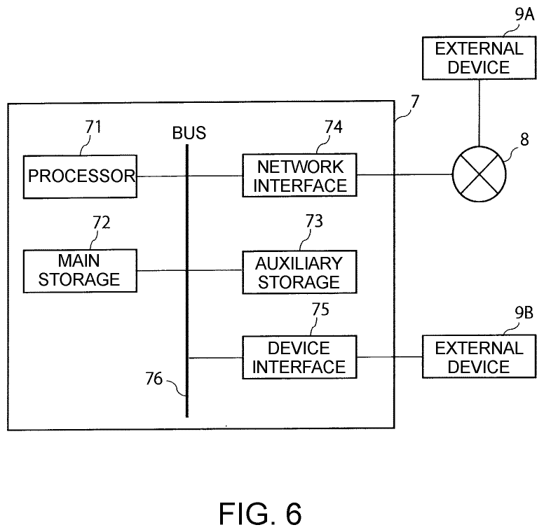

FIG. 6 is a block diagram illustrating an example of a hardware configuration of each device (at least one of the first information processing device 10 or the second information processing device 20) in the above embodiments. As an example, each device may be implemented as a computer 7 provided with a processor 71, a main storage device 72, an auxiliary storage device 73, a network interface 74, and a device interface 75, which are connected via a bus 76.

The computer 7 of FIG. 6 is provided with each component one by one but may be provided with a plurality of the same components. Although one computer 7 is illustrated in FIG. 6, the software may be installed on a plurality of computers, and each of the plurality of computer may execute the same or a different part of the software processing. In this case, it may be in a form of distributed computing where each of the computers communicates with each of the computers through, for example, the network interface 74 to execute the processing. That is, each device (at least one of the first information processing device 10 or the second information processing device 20) in the above embodiments may be configured as a system where one or more computers execute the instructions stored in one or more storages to enable functions. Each device may be configured such that the information transmitted from a terminal is processed by one or more computers provided on a cloud and results of the processing are transmitted to the terminal.

Various arithmetic operations of each device (at least one of the first information processing device 10 or the second information processing device 20) in the above embodiments may be executed in parallel processing using one or more processors or using a plurality of computers over a network. The various arithmetic operations may be allocated to a plurality of arithmetic cores in the processor and executed in parallel processing. Some or all the processes, means, or the like of the present disclosure may be implemented by at least one of the processors or the storage devices provided on a cloud that can communicate with the computer 7 via a network. Thus, each device in the above embodiments may be in a form of parallel computing by one or more computers.

The processor 71 may be an electronic circuit (such as, for example, a processor, processing circuitry, processing circuitry, CPU, GPU, FPGA, or ASIC) that executes at least controlling the computer or arithmetic calculations. The processor 71 may also be, for example, a general-purpose processing circuit, a dedicated processing circuit designed to perform specific operations, or a semiconductor device which includes both the general-purpose processing circuit and the dedicated processing circuit. Further, the processor 71 may also include, for example, an optical circuit or an arithmetic function based on quantum computing.

The processor 71 may execute an arithmetic processing based on data and/or a software input from, for example, each device of the internal configuration of the computer 7, and may output an arithmetic result and a control signal, for example, to each device. The processor 71 may control each component of the computer 7 by executing, for example, an OS (Operating System), or an application of the computer 7.

Each device (at least one of the first information processing device 10 or the second information processing device 20) in the above embodiments may be enabled by one or more processors 71. The processor 71 may refer to one or more electronic circuits located on one chip, or one or more electronic circuitries arranged on two or more chips or devices. In the case of a plurality of electronic circuitries are used, each electronic circuit may communicate by wired or wireless.

The main storage device 72 may store, for example, instructions to be executed by the processor 71 or various data, and the information stored in the main storage device 72 may be read out by the processor 71. The auxiliary storage device 73 is a storage device other than the main storage device 72. These storage devices shall mean any electronic component capable of storing electronic information and may be a semiconductor memory. The semiconductor memory may be either a volatile or non-volatile memory. The storage device for storing various data or the like in each device (at least one of the first information processing device 10 or the second information processing device 20) in the above embodiments may be enabled by the main storage device 72 or the auxiliary storage device 73 or may be implemented by a built-in memory built into the processor 71. For example, the storages 102 in the above embodiments may be implemented in the main storage device 72 or the auxiliary storage device 73.

In the case of each device (at least one of the first information processing device 10 or the second information processing device 20) in the above embodiments is configured by at least one storage device (memory) and at least one of a plurality of processors connected/coupled to/with this at least one storage device, at least one of the plurality of processors may be connected to a single storage device. Or at least one of the plurality of storages may be connected to a single processor. Or each device may include a configuration where at least one of the plurality of processors is connected to at least one of the plurality of storage devices. Further, this configuration may be implemented by a storage device and a processor included in a plurality of computers. Moreover, each device may include a configuration where a storage device is integrated with a processor (for example, a cache memory including an L1 cache or an L2 cache).

The network interface 74 is an interface for connecting to a communication network 8 by wireless or wired. The network interface 74 may be an appropriate interface such as an interface compatible with existing communication standards. With the network interface 74, information may be exchanged with an external device 9A connected via the communication network 8. Note that the communication network 8 may be, for example, configured as WAN (Wide Area Network), LAN (Local Area Network), or PAN (Personal Area Network), or a combination of thereof, and may be such that information can be exchanged between the computer 7 and the external device 9A. The internet is an example of WAN, IEEE802.11 or Ethernet (registered trademark) is an example of LAN, and Bluetooth (registered trademark) or NFC (Near Field Communication) is an example of PAN.

The device interface 75 is an interface such as, for example, a USB that directly connects to the external device 9B.

The external device 9A is a device connected to the computer 7 via a network. The external device 9B is a device directly connected to the computer 7.

The external device 9A or the external device 9B may be, as an example, an input device. The input device is, for example, a device such as a camera, a microphone, a motion capture, at least one of various sensors, a keyboard, a mouse, or a touch panel, and gives the acquired information to the computer 7. Further, it may be a device including an input unit such as a personal computer, a tablet terminal, or a smartphone, which may have an input unit, a memory, and a processor.

The external device 9A or the external device 9B may be, as an example, an output device. The output device may be, for example, a display device such as, for example, an LCD (Liquid Crystal Display), or an organic EL (Electro Luminescence) panel, or a speaker which outputs audio. Moreover, it may be a device including an output unit such as, for example, a personal computer, a tablet terminal, or a smartphone, which may have an output unit, a memory, and a processor.

Further, the external device 9A or the external device 9B may be a storage device (memory). The external device 9A may be, for example, a network storage device, and the external device 9B may be, for example, an HDD storage.

Furthermore, the external device 9A or the external device 9B may be a device that has at least one function of the configuration element of each device (at least one of the first information processing device 10 or the second information processing device 20) in the above embodiments. That is, the computer 7 may transmit a part of or all of processing results to the external device 9A or the external device 9B, or receive a part of or all of processing results from the external device 9A or the external device 9B.

In the present specification (including the claims), the representation (including similar expressions) of “at least one of a, b, and c” or “at least one of a, b, or c” includes any combinations of a, b, c, a-b, a-c, b-c, and a-b-c. It also covers combinations with multiple instances of any element such as, for example, a-a, a-b-b, or a-a-b-b-c-c. It further covers, for example, adding another element d beyond a, b, and/or c, such that a-b-c-d.

In the present specification (including the claims), the expressions such as, for example, “data as input,” “using data,” “based on data,” “according to data,” or “in accordance with data” (including similar expressions) are used, unless otherwise specified, this includes cases where data itself is used, or the cases where data is processed in some ways (for example, noise added data, normalized data, feature quantities extracted from the data, or intermediate representation of the data) are used. When it is stated that some results can be obtained “by inputting data,” “by using data,” “based on data,” “according to data,” “in accordance with data” (including similar expressions), unless otherwise specified, this may include cases where the result is obtained based only on the data, and may also include cases where the result is obtained by being affected factors, conditions, and/or states, or the like by other data than the data. When it is stated that “output/outputting data” (including similar expressions), unless otherwise specified, this also includes cases where the data itself is used as output, or the cases where the data is processed in some ways (for example, the data added noise, the data normalized, feature quantity extracted from the data, or intermediate representation of the data) is used as the output.

In the present specification (including the claims), when the terms such as “connected (connection)” and “coupled (coupling)” are used, they are intended as non-limiting terms that include any of “direct connection/coupling,” “indirect connection/coupling,” “electrically connection/coupling,” “communicatively connection/coupling,” “operatively connection/coupling,” “physically connection/coupling,” or the like. The terms should be interpreted accordingly, depending on the context in which they are used, but any forms of connection/coupling that are not intentionally or naturally excluded should be construed as included in the terms and interpreted in a non-exclusive manner.

In the present specification (including the claims), when the expression such as “A configured to B,” this may include that a physically structure of A has a configuration that can execute operation B, as well as a permanent or a temporary setting/configuration of element A is configured/set to actually execute operation B. For example, when the element A is a general-purpose processor, the processor may have a hardware configuration capable of executing the operation B and may be configured to actually execute the operation B by setting the permanent or the temporary program (instructions). Moreover, when the element A is a dedicated processor, a dedicated arithmetic circuit, or the like, a circuit structure of the processor or the like may be implemented to actually execute the operation B, irrespective of whether or not control instructions and data are actually attached thereto.

In the present specification (including the claims), when a term referring to inclusion or possession (for example, “comprising/including,” “having,” or the like) is used, it is intended as an open-ended term, including the case of inclusion or possession an object other than the object indicated by the object of the term. If the object of these terms implying inclusion or possession is an expression that does not specify a quantity or suggests a singular number (an expression with a or an article), the expression should be construed as not being limited to a specific number.

In the present specification (including the claims), although when the expression such as “one or more,” “at least one,” or the like is used in some places, and the expression that does not specify a quantity or suggests a singular number (the expression with a or an article) is used elsewhere, it is not intended that this expression means “one.” In general, the expression that does not specify a quantity or suggests a singular number (the expression with a or an as article) should be interpreted as not necessarily limited to a specific number.

In the present specification, when it is stated that a particular configuration of an example results in a particular effect (advantage/result), unless there are some other reasons, it should be understood that the effect is also obtained for one or more other embodiments having the configuration. However, it should be understood that the presence or absence of such an effect generally depends on various factors, conditions, and/or states, etc., and that such an effect is not always achieved by the configuration. The effect is merely achieved by the configuration in the embodiments when various factors, conditions, and/or states, etc., are met, but the effect is not always obtained in the claimed invention that defines the configuration or a similar configuration.

In the present specification (including the claims), when the term such as “maximize/maximization” is used, this includes finding a global maximum value, finding an approximate value of the global maximum value, finding a local maximum value, and finding an approximate value of the local maximum value, should be interpreted as appropriate accordingly depending on the context in which the term is used. It also includes finding on the approximated value of these maximum values probabilistically or heuristically. Similarly, when the term such as “minimize/minimization” is used, this includes finding a global minimum value, finding an approximated value of the global minimum value, finding a local minimum value, and finding an approximated value of the local minimum value, and should be interpreted as appropriate accordingly depending on the context in which the term is used. It also includes finding the approximated value of these minimum values probabilistically or heuristically. Similarly, when the term such as “optimize/optimization” is used, this includes finding a global optimum value, finding an approximated value of the global optimum value, finding a local optimum value, and finding an approximated value of the local optimum value, and should be interpreted as appropriate accordingly depending on the context in which the term is used. It also includes finding the approximated value of these optimal values probabilistically or heuristically.

In the present specification (including claims), when a plurality of hardware performs a predetermined process, the respective hardware may cooperate to perform the predetermined process, or some hardware may perform all the predetermined process. Further, a part of the hardware may perform a part of the predetermined process, and the other hardware may perform the rest of the predetermined process. In the present specification (including claims), when an expression (including similar expressions) such as “one or more hardware perform a first process and the one or more hardware perform a second process,” or the like, is used, the hardware that perform the first process and the hardware that perform the second process may be the same hardware, or may be the different hardware. That is: the hardware that perform the first process and the hardware that perform the second process may be included in the one or more hardware. Note that, the hardware may include an electronic circuit, a device including the electronic circuit, or the like.

In the present specification (including the claims), when a plurality of storage devices (memories) store data, an individual storage device among the plurality of storage devices may store only a part of the data or may store the entire data. Further, some storage devices among the plurality of storage devices may include a configuration for storing data.

The above mentioned embodiments would be summarized as follows:

(1) An information processing system comprising at least a first information processing device and a second information processing device,
the second information processing device configured to
   transmit data relating to an atomic structure to the first information processing device,
the first information processing device configured to:
   receive the data relating to the atomic structure from the second information processing device;
   input the data relating to the atomic structure into a first model;
   acquire first information based on an output from an intermediate layer of the first model; and
   transmit the first information to the second information processing device, and
the second information processing device configured to
   input the first information received from the first information processing device into a second model to acquire a predetermined value for the atomic structure.

The data relating to the atomic structure is a concept that includes data regarding atomic structure. Further, the first information may include at least values of output of the intermediate layer itself or converted values of the output of the intermediate layer by predetermined processing.

(2) The information processing system according to (1), wherein the first information processing device is configured to: further acquire second information based on an output from an output layer of the first model; and transmit the second information to the second information processing device.

The second information may include at least values of output of the output layer itself or converted values of the output of the output layer by predetermined processing.

(3) The information processing system according to (1) or (2), wherein the first information processing device is configured to: execute backward propagation processing of the first model to further acquire third information; and transmit the third information to the second information processing device.

(4) The information processing system according to any one of (1) to (3), wherein the predetermined value is a physical property value for the atomic structure.

(5) The information processing system according to (4), wherein the physical property value is energy for the atomic structure.

(6) The information processing system according to any one of (1) to (5), wherein the predetermined value is a potential parameter.

(7) The information processing system according to any one of (1) to (6), wherein the first model is a model which forms a Neural Network Potential (NNP).

(8) The information processing system according to any one of (1) to (7), wherein the second information processing device is configured to: acquire information about at least part of the first model from the first information processing device; and execute backward propagation processing using the acquired information about at least part of the first model and an output from an output layer of the second model.

(9) The information processing system according to any one of (1) to (8), wherein the second information processing device is configured to calculate similarity between at least part of the atomic structure and at least part of another atomic structure, based on the received first information.

(10) The information processing system according to any one of (1) to (9), wherein the second information processing device is configured to cause a display device to display the first information.

(11) An information processing device, comprising:
at least one memory; and
at least one processor,
the at least one processor configured to:
   transmit data relating to an atomic structure to another information processing device;
   acquire first information from the other information processing device; and
   acquire a predetermined value for the atomic structure by inputting the first information into a second model, wherein
the first information is information based on an output from an intermediate layer of a first model which is generated by the other information processing device inputting the data relating to the atomic structure into the first model.

The information based on the output from the intermediate layer may include at least values of output of the intermediate layer itself or converted output values of the intermediate layer by a predetermined process.

(12) The information processing device according to (11), wherein the predetermined value is a physical property value for the atomic structure.

(13) The information processing device according to (11) or (12), wherein the physical property value is energy for the atomic structure.

(14) The information processing device according to (12) or (13), wherein the at least one processor is configured to: acquire information about at least part of the first model from the other information processing device; and execute backward propagation processing using the received information about at least part of the first model and an output from an output layer of the second model.

(15) The information processing device according to any one of (11) to (14), wherein the at least one processor is configured to calculate similarity between at least part of the atomic structure and at least part of another atomic structure, based on the received first information.

(16) The information processing device according to any one of (11) to (15), wherein the predetermined value is a potential parameter.

(17) The information processing device according to any one of (11) to (16) wherein the at least one processor is configured to cause a display device to display the first information.

(18) The information processing device according to (17), wherein the first information displayed on the display device is information lower in dimension than the output from the intermediate layer of the first model.

(19) The information processing device according to any one of (11) to (18), wherein the first model is a model which forms the NNP.

(20) An information processing method comprising:
transmitting, by at least one processor, data relating to an atomic structure to another information processing device;
acquiring, by the at least one processor, first information from the other information processing device; and
acquiring, by the at least one processor, a predetermined value for the atomic structure by inputting the first information into a second model, wherein
the first information is information based on an output from an intermediate layer of a first model which is generated by the other information processing device inputting the data relating to the atomic structure into the first model.

While certain embodiments of the present disclosure have been described in detail above, the present disclosure is not limited to the individual embodiments described above. Various additions, changes, substitutions, partial deletions, etc. are possible to the extent that they do not deviate from the conceptual idea and purpose of the present disclosure derived from the contents specified in the claims and their equivalents. For example, when numerical values or mathematical formulas are used in the description in the above-described embodiments, they are shown for illustrative purposes only and do not limit the scope of the present disclosure. Further, the order of each operation shown in the embodiments is also an example, and does not limit the scope of the present disclosure.

1. An information processing system comprising at least a first information processing device and a second information processing device,
the second information processing device configured to
transmit data relating to an atomic structure to the first information processing device,
the first information processing device configured to:
receive the data relating to the atomic structure from the second information processing device;
input the data relating to the atomic structure into a first model;
acquire first information based on an output from an intermediate layer of the first model; and
transmit the first information to the second information processing device, and
the second information processing device configured to
input the first information received from the first information processing device into a second model to acquire a predetermined value for the atomic structure.

2. The information processing system according to claim 1, wherein the first information processing device is configured to:
further acquire second information based on an output from an output layer of the first model; and
transmit the second information to the second information processing device.

3. The information processing system according to claim 1, wherein the first information processing device is configured to:
execute backward propagation processing of the first model to further acquire third information; and
transmit the third information to the second information processing device.

4. The information processing system according to claim 1, wherein the predetermined value is a physical property value for the atomic structure.

5. The information processing system according to claim 4, wherein the physical property value is energy for the atomic structure.

6. The information processing system according to claim 1, wherein the predetermined value is a potential parameter.

7. The information processing system according to claim 1, wherein the first model is a model which forms a Neural Network Potential (NNP).

8. The information processing system according to claim 1, wherein the second information processing device is configured to:
acquire information about at least part of the first model from the first information processing device; and
execute backward propagation processing using the acquired information about at least part of the first model and an output from an output layer of the second model.

9. The information processing system according to claim 1, wherein the second information processing device is configured to calculate similarity between at least part of the atomic structure and at least part of another atomic structure, based on the received first information.

10. The information processing system according to claim 1, wherein the second information processing device is configured to cause a display device to display the first information.

11. An information processing device, comprising:
at least one memory; and
at least one processor configured to:
transmit data relating to an atomic structure to another information processing device;
acquire first information from the other information processing device; and
acquire a predetermined value for the atomic structure by inputting the first information into a second model, wherein
the first information is information based on an output from an intermediate layer of a first model which is generated by the other information processing device inputting the data relating to the atomic structure into the first model.

12. The information processing device according to claim 11, wherein the predetermined value is a physical property value for the atomic structure.

13. The information processing device according to claim 11, wherein the physical property value is energy for the atomic structure.

14. The information processing device according to claim 12, wherein the at least one processor is configured to:
acquire information about at least part of the first model from the other information processing device; and
execute backward propagation processing using the received information about at least part of the first model and an output from an output layer of the second model.

15. The information processing device according to claim 11, wherein the at least one processor is configured to calculate similarity between at least part of the atomic structure and at least part of another atomic structure, based on the received first information.

16. The information processing device according to claim 11, wherein the predetermined value is a potential parameter.

17. The information processing device according to claim 11, wherein the at least one processor is configured to cause a display device to display the first information.

18. The information processing device according to claim 17, wherein the first information displayed on the display device is information lower in dimension than the output from the intermediate layer of the first model.

19. The information processing device according to claim 11, wherein the first model is a model which forms the NNP.

20. An information processing method comprising:
transmitting, by at least one processor, data relating to an atomic structure to another information processing device;
acquiring, by the at least one processor, first information from the other information processing device; and
acquiring, by the at least one processor, a predetermined value for the atomic structure by inputting the first information into a second model, wherein
the first information is information based on an output from an intermediate layer of a first model which is generated by the other information processing device inputting the data relating to the atomic structure into the first model.
